(19) United States
(12) Patent Application Publication
(10) Pub. No.: US 2013/0125882 A1
(43) Pub. Date: May 23, 2013

(54) METHOD AND COMPOSITION FOR TREATING ASTHMA AND COPD

(71) Applicant: Jonathan Matz, Columbia, MD (US)

(72) Inventor: Jonathan Matz, Columbia, MD (US)

(21) Appl. No.: 13/298,543

(22) Filed: Nov. 17, 2011

(51) Int. Cl. A61K 31/573; A61K 31/56; A61P 11/06; A61M 16/14; A61B 19/02; A61M 11/04; A61M 15/00; A61K 31/58; A61P 11/00

(52) U.S. Cl. 128/200.23; 514/171; 206/438; 128/203.15; 128/203.12

(57) ABSTRACT

A pharmaceutical composition and method for the treatment of asthma or other respiratory disease by inhalation from a pressurized metered dose inhaler (pMDI), a dry powder inhaler (DPI) or nebulizer, the composition and method being based on a combination of a short acting beta agonist (SABA) and an inhaled corticosteroid (ICS) in a form adapted to be delivered for inhalation from the pMDI, DPI or nebulizer.

### BACKGROUND OF THE INVENTION

1. Field of the Invention

Aspects of the present invention relate to compositions suitable for the treatment of asthma and chronic obstructive pulmonary diseases (COPD) by inhalation therapy.

2. Description of the Related Art

The entire contents and disclosures of each publication, reference, patent and other prior art referred to herein are incorporated by reference.

Administration via inhalation of long-acting corticosteroids (ICS) in conjunction with long-acting beta-agonists (LABA) has been available for years for the treatment of asthma and chronic obstructive pulmonary diseases, commonly abbreviated as COPD, examples of such being emphysema and chronic bronchitis. For example, the combination of budesonide (an ICS) and formoterol (a LABA) is available under the brand name Symbicort® and is recommended by the National Asthma Education and Prevention Program of the National Institute of Health for long-term control and prevention of symptoms of moderate and severe persistent asthma. The combination is offered in a dry powder inhaler device marketed as ADVAIR Diskus® by GlaxoSmithKline. Budesonide is also marketed worldwide by AstraZeneca under the name Pulmircort®.

Chronic obstructive pulmonary disease (COPD) is the fourth leading cause of death in the United States. In 2002 alone, COPD claimed over 120,000 lives. More than 10.7 million adult Americans are thought to have COPD, and approximately 24 million others are thought to have impaired lung function, which is a possible early sign of COPD. Women are more than twice as likely to develop COPD as men, and in 2002, more women than men died due to COPD (61,000 vs. 59,000). The medical costs associated with COPD can be astronomic. In 2004, COPD cost the U.S. health care system an estimated $37.2 billion, including direct medical costs of $20.9 billion, $7.4 billion in indirect morbidity, and $8.9 billion in indirect mortality costs. Although no available drugs halt the long-term decline of lung function associated with COPD, bronchodilators reduce the frequency and severity of exacerbations, improve health status, and improve exercise tolerance. As a class, bronchodilators improve forced expiratory volume in 1 second (FEV1) and some other spirometric parameters by reducing smooth muscle tone. Bronchodilators can also reduce air trapping and hyperinflation, two of the key pathophysiologic abnormalities seen in patients with COPD, at rest and during physical activity.

Asthma is an inflammatory disease of the lower airways characterized by reversible airway obstruction and bronchial hyper-responsiveness. It is one of the most common diseases in industrialized countries, affecting approximately 130 million people globally. There were 17 million diagnosed asthma sufferers in the USA in 2001.

Other examples of respiratory diseases where inflammation may play a role include: eosinophilic cough, bronchitis, sarcoidosis, pulmonary fibrosis, pulmonary hypertension, cystic fibrosis, and bronchiectasis. Asthma is defined by airway inflammation, reversible obstruction and airway hyperresponsiveness. In this disease the inflammatory cells that are involved are predominantly eosinophils, T lymphocytes and mast cells, although neutrophils and macrophages may also be important. Vast arrays of cytokines and chemokines have been shown to be increased in the airways and play a role in the pathophysiology of this disease by promoting inflammation, obstruction and hyperresponsiveness.

Acute bronchitis is an acute disease that occurs during an infection or irritating event for example by pollution, dust, gas or chemicals, of the lower airways. Chronic bronchitis is defined by the presence of cough and phlegm production on most days for at least 3 months of the year, for 2 years. One can also find during acute or chronic bronchitis within the airways inflammatory cells, mostly neutrophils, with a broad array of chemokines and cytokines. These mediators are thought to play a role in the inflammation, symptoms and mucus production that occur with this disease.

Eosinophilic cough is characterized by chronic cough and the presence of inflammatory cells, mostly eosinophils, within the airways of patients in the absence of airway obstruction or hyperresponsiveness. Several cytokines and chemokines are increased in this disease, although they are mostly eosinophil directed. Eosinophils are recruited and activated within the airways and potentially release enzymes and oxygen radicals that play a role in the perpetuation of inflammation and cough.

Sarcoidosis is a disease of unknown cause where chronic non-caseating granulomas occur within tissue. The lung is the organ most commonly affected. Lung bronchoalveolar lavage shows an increase in lymphocytes, macrophages and sometimes neutrophils and eosinophils. These cells are also recruited and activated by cytokines or chemokines and may be involved in the pathogenesis of the disease.

Pulmonary fibrosis is a disease of lung tissue characterized by progressive and chronic fibrosis (scarring) that will lead to chronic respiratory insufficiency. Different types and causes of pulmonary fibrosis exist but all are characterized by inflammatory cell influx and persistence, activation and proliferation of fibroblasts with collagen deposition in lung tissue. These events seem related to the release of cytokines and chemokines within lung tissue.

### SUMMARY OF THE INVENTION

Aspects of the subject invention concern a pharmaceutical composition for the treatment of a respiratory disease associated with inflammation in human patients by inhalation from a pressurized metered dose inhaler (pMDI), a dry powder inhaler (DPI) or a nebulizer, the composition consisting essentially of [1] an inhaled corticosteroid (ICS) and [2] a short acting beta agonist (SABA) in a form adapted to be delivered for inhalation from a pMDI, a DPI or a nebulizer at a dose of 1 to 500 mcg (microgram) of [1] and a dose of 1 to 250 mcg of [2] per delivery.

Aspects of the subject invention also relate to a method for treating a respiratory disease associated with inflammation in human patients comprising the administration to a patient in need thereof, a therapeutically effective amount of the above-described pharmaceutical composition by inhalation from a pressurized metered dose inhaler (pMDI), a dry powder inhaler (DPI) or a nebulizer.

Another embodiment of the invention comprises a pressurized metered dose inhaler (pMDI), a dry powder inhaler (DPI) or a nebulizer containing the above-described pharmaceutical composition

### DETAILED DESCRIPTION OF THE EMBODIMENTS

Aspects of the present invention are predicated on the discovery that certain combinations of inhaled corticosteroids (ICS) and short acting beta agonists (SABA) offer significant advantages when administered by inhalation from a pressurized metered dose inhaler (pMDI), a dry powder inhaler (DPI) or a nebulizer not heretofore appreciated by those skilled in the art.

Physicians treating asthma and COPDs have observed that sufferers thereof will use SABAs for relief when their condition flares, often over-using the product when they should also be using in combination therewith, an anti-inflammatory therapy such as an ICS. Aspects of the present invention are highly advantageous in that it enables an enhanced therapeutic effect in those patients who are poorly compliant with ICS, who overuse SABA during flare-ups of their conditions, and those with mild asthma that use SABAs on an ‘as needed’ basis.

More particularly, care givers fear the overuse of albuterol and other SABA's during asthma flare-ups and patients often forget or do not know to use an inhaled steroid if their symptoms deteriorate. Aspects of the present invention address these fears by ensuring that patients needing increased SABA for symptoms will be simultaneously receiving anti-inflammatory therapy. Thus, the invention provides the added benefit over and above the use of albuterol and other SABAs alone for prophylactic and recurring use in persistent asthma, intermittent asthma and exercise induced bronchospasm/asthma.

Moreover, it is known that an undesired side-effect (tachyphylaxis) is associated with the prolonged use of LABAs (long acting beta agonist/bronchodilator); however, it has not been shown that the composition of the invention used on an as needed basis gives rise to this highly disadvantageous side-effect.

Utilizing the composition of the invention on an “as needed” basis (2 puffs every 4 hours) will accomplish the goal of asthma symptom control with less overall ICS than if the patient took the ICS product daily as is the current standard practice in the National Heart, Lung, and Blood Institute (NHLBI)—Step 1, Step 2, and Step 3 protocol for treating asthma.

Aspects of the present invention further offer the added benefit of allowing for the on demand use of a combination product without the increased rate of death found in connection with the ICS/LABA (long acting bronchodilators) for use as reliever therapy in asthma exacerbations (O'Byrne—Am J Resp Crit Care Med 2005;171:129-36 and Rabe—Lancet 2006;368;744-53). Finally, aspects of the present invention greatly improve patient compliance since only a single payment (or co-pay) is required, thus ensuring anti-inflammatory therapy when the administration of a SABA is required.

Administration may be by inhalation orally or intranasally. The active ingredients are preferably adapted to be administered, either together or individually, from dry powder inhaler(s) (DPIs), especially Turbuhaler®. (Astra AB), pressurized metered dose inhaler(s) (pMDIs), or nebulizer(s). It will be understood by those skilled in the art that the composition may be administered by any suitable such device provided that it is provided with a mechanism operable to actuate the device to deliver the appropriate dose of the medicament of the invention.

Suitable pMDIs for administration of the compositions of the invention are those disclosed in U.S. Pat. Nos. 6,860,262; 7,836,880; and 7,740,463. Pressurized metered-dose inhalers (pMDI) are devices that deliver a specific amount of medication to the lungs, in the form of a short burst of aerosolized medicine that is inhaled by the patient. It is the most commonly used delivery system for treating asthma and COPD. Hydrofluoroalkanes (HFA), such as either HFA 134a (1,1,1,2,-tetrafluoroethane) or HFA 227 (1,1,1,2,3,3,3-heptafluoropropane) or combinations of the two are the most commonly used aerosols; however, it will be understood by those skilled in the art that any suitable aerosol may be employed in the practice of the invention.

Suitable DPIs for administration of the compositions of the invention are those disclosed in U.S. Pat. Nos. 7,987,845; 7,958,890; and 7,896,005.

Suitable nebulizers for administration of the compositions of the invention are those disclosed in U.S. Pat. Nos. 6,962,151; 8,015,969; and 8,001,963.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The pharmaceutical compositions of the invention include a “therapeutically effective amount” or a “prophylactically effective amount” of one or more of the compounds of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the compound may vary according to factors such as the disease state, age, sex, and weight of the individual, and the ability of the therapeutic compound to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Delivery of the compound of the present invention to the lung by way of inhalation is an important method of treating a variety of respiratory conditions (airway inflammation) noted throughout the specification, including such common local conditions as bronchial asthma and chronic obstructive pulmonary disease. The compound can be administered to the lung in the form of an aerosol of particles of respirable size (less than about 10 μm in diameter). The aerosol formulation can be presented as a liquid or a dry powder. In order to assure proper particle size in a liquid aerosol, as a suspension, particles can be prepared in respirable size and then incorporated into the suspension formulation containing a propellant. Alternatively, formulations can be prepared in solution form in order to avoid the concern for proper particle size in the formulation. Solution formulations should be dispensed in a manner that produces particles or droplets of respirable size.

The following are specific illustrative examples of the invention wherein the total amounts of each product will depend on the size of the canister:

1. Combination of beclomethasone diproprionate [50 or 100 mcg from the valve] with albuterol sulfate [108 mcg from the valve] in the propellant HFA-134a (1,1,1,2 tetrafluoroethane) and ethanol in a pMDI.

2. Combination of beclomethasone diproprionate [50 or 100 mcg from the valve] with levalbuterol tartrate [59 mcg from the valve] in the propellant HFA-134a (1,1,1,2 tetrafluoroethane) and Dehydrated Alcohol USP and Oleic Acid NF in a pMDI.

3. Combination of fluticasone proprionate [50, 125, 250 mcg from the valve] or fluticasone furoate [at doses of 50 to 250 mcg from the valve] with albuterol sulfate [108 mcg from the valve] in the propellant HFA-134a (1,1,1,2 tetrafluoroethane) and ethanol in a pMDI.

4. Combination of fluticasone proprionate [50, 125, 250 mcg from the valve] or fluticasone furoate [at doses of 50 to 250 mcg from the valve] with levalbuterol tartrate [59 mcg from the valve] in the propellant HFA-134a (1,1,1,2 tetrafluoroethane) and Dehydrated Alcohol USP and Oleic Acid NF in a pMDI.

5. Combination of ciclesonide [100 or 200 mcg from the valve] with albuterol sulfate [108 mcg from the valve] in the propellant HFA-134a (1,1,1,2 tetrafluoroethane) and ethanol in a pMDI.

6. Combination of ciclesonide [100 or 200 mcg from the valve] with levalbuterol tartrate [59 mcg from the valve] in the propellant HFA-134a (1,1,1,2 tetrafluoroethane) and Dehydrated Alcohol USP and Oleic Acid NF in a pMDI.

7. Combination of mometasone furoate [115 or 225 mcg from the valve] with albuterol sulfate [108 mcg from the valve] in the propellant HFA-134a (1,1,1,2 tetrafluoroethane) and ethanol in a pMDI.

8. Combination of mometasone furoate [115 or 225 mcg from the valve] with levalbuterol tartrate [59 mcg from the valve] in the propellant HFA-134a (1,1,1,2 tetrafluoroethane) and Dehydrated Alcohol USP and Oleic Acid NF in a pMDI.

9. Combination of budesonide [91 or 181 mcg from the valve] with albuterol sulfate [108 mcg from the valve] in the propellant HFA-134a (1,1,1,2 tetrafluoroethane) and ethanol in a pMDI.

10. Combination of budesonide [91 or 181 mcg from the valve] with levalbuterol tartrate [59 mcg from the valve] in the propellant HFA-134a (1,1,1,2 tetrafluoroethane) and Dehydrated Alcohol USP and Oleic Acid NF in a pMDI.

11. Combination of a microcrystalline suspension of triamcinolone acetonide 100 or 200 mcg with albuterol sulfate [108 mcg from the valve] in the propellant HFA-134a (1,1,1,2 tetrafluoroethane) and ethanol in a pMDI.

12. Combination of a microcrystalline suspension of triamcinolone acetonide 100 or 200 mcg [from the valve] with levalbuterol tartrate [59 mcg from the valve] in the propellant HFA-134a (1,1,1,2 tetrafluoroethane) and Dehydrated Alcohol USP and Oleic Acid NF in a pMDI.

13. Combination of a microcrystalline suspension of flunisolide 100 or 250 mcg [from the valve] as the hemihydrate with albuterol sulfate [108 mcg from the valve] in the propellant HFA-134a (1,1,1,2 tetrafluoroethane) and ethanol in a pMDI.

14. Combination of a microcrystalline suspension of flunisolide 100 or 250 mcg [from the valve] as the hemihydrates with levalbuterol tartrate [59 mcg from the valve] in the propellant HFA-134a (1,1,1,2 tetrafluoroethane) and Dehydrated Alcohol USP and Oleic Acid NF in a pMDI.

Those skilled in the art will be aware that similar combinations can be made for dry powder inhalation at the ratios noted for the pMDIs but would not include the propellant (HFA-134a, or HFA 227) or the alcohol, ethanol or oleic acid. Rather, anhydrous lactose may be utilized.

What is claimed is:

1. A pharmaceutical composition for the treatment of asthma or other respiratory disease by inhalation from a pressurized metered dose inhaler (pMDI), a dry powder inhaler (DPI) or nebulizer, said composition consisting essentially of [1] an inhaled corticosteroid (ICS) and [2] a short acting beta agonist (SABA) in a form adapted to be delivered for inhalation from said pMDI, DPI or nebulizer at a dose of 1 to 500 mcg of [1] and a dose of 1 to 250 mcg of [2] per delivery.

2. The pharmaceutical composition of claim 1, wherein said delivery is from a pMDI.

3. The pharmaceutical composition of claim 1, wherein said delivery is from a DPI.

4. The pharmaceutical composition of claim 1, wherein said delivery is from a nebulizer.

5. The pharmaceutical composition of claim 1, wherein said SABA is salbutamol (albuterol), levalbuterol, pirbuterol, terbutaline, epinephrine, a salt thereof with a pharmaceutically acceptable acid or a mixture thereof.

6. The pharmaceutical composition of claim 4, wherein said SABA is salbutamol (albuterol) and said dose is 108 mcg.

7. The pharmaceutical composition of claim 4, wherein said SABA is levalbuterol and said dose is 59 mcg.

8. The pharmaceutical composition of claim 1, wherein said ICS is beclomethasone diproprionate, fluticasone furoate or fluticasone proprionate, ciclesonide, mometasone furoate, budesonide, flunisolide, or triamcinolone or a mixture thereof.

9. The pharmaceutical composition of claim 8, wherein said ICS is beclomethasone diproprionate and said dose is 50 or 100 mcg.

10. The pharmaceutical composition of claim 8, wherein said ICS is fluticasone proprionate or fluticasone furoate and said dose is 25, 50, 125 or 250 mcg.

11. The pharmaceutical composition of claim 8, wherein said ICS is ciclesonide and said dose is 100 or 200 mcg.

12. The pharmaceutical composition of claim 8, wherein said ICS is mometasone furoate and said dose is 115 or 225 mcg.

13. The pharmaceutical composition of claim 8, wherein said ICS is budesonide and said dose is 91 or 181 mcg.

14. The pharmaceutical composition of claim 8, wherein said ICS is flunisolide and said dose is 100 or 250 mcg.

15. The pharmaceutical composition of claim 8, wherein said ICS is triamcinolone and said dose is 100, 200 or 400 mcg.

16. A method for treating a respiratory disease associated with inflammation in human patients comprising the administration to a patient in need thereof a therapeutically effective amount of the pharmaceutical composition of claim 1 by inhalation from a pressurized metered dose inhaler (pMDI), a dry powder inhaler (DPI) or a nebulizer.

17. A pressurized metered dose inhaler (pMDI), a dry powder inhaler (DPI) or a nebulizer containing the pharmaceutical composition of claim 1.

18. An article of manufacture comprising packaging material and a pharmaceutical agent contained within said packaging material wherein said packaging material comprises a label which indicates that said pharmaceutical agent can be used for treating a subject suffering from a respiratory disease associated with inflammation in human patients, and wherein said pharmaceutical agent is a composition according to claim 1.

19. An article of manufacture according to claim 18, wherein said packaging material is a pressurized metered dose inhaler (pMDI).

20. An article of manufacture according to claim 18, wherein said packaging material is a dry powder inhaler (DPI).

21. An article of manufacture according to claim 18, wherein said packaging material is a nebulizer.
